(19) United States
(12) Patent Application Publication
(10) Pub. No.: US 2019/0021595 A1
(43) Pub. Date: Jan. 24, 2019

(54) Device For Obtaining At Least One Physiological Parameter

(71) Applicant: BEWELLCONNECT, Paris (FR)

(72) Inventor: Eric Sebban, Paris (FR)

(73) Assignee: BEWELLCONNECT, Paris (FR)

(21) Appl. No.: 16/068,225

(22) PCT Filed: Jan. 5, 2017
(86) PCT No.: PCT/EP2017/050223

§ 371 Date: Jul. 5, 2018

(30) Foreign Application Priority Data
Jan. 5, 2016 (FR) 1650053

(51) Int. Cl. A61B 5/00; A61B 5/024; A61B 5/0205

(52) U.S. Cl. CPC A61B 5/0008; A61B 5/02438; A61B 5/02055; A61B 5/01; A61B 5/0006; A61B 5/02405; A61B 5/742; A61B 5/0022

(57) ABSTRACT

A device for obtaining at least one physiological parameter of an individual, the device being a portable device comprising: body temperature measuring means (200) of the individual, display means configured to display the physiological parameter, the physiological parameter comprising the body temperature, communication means, for example with a communications network to which equipment is connected, in particular wireless, and processing means configured to transmit the physiological parameter to the equipment via the network.

### TECHNICAL FIELD

This relates to the technical field of devices for obtaining a physiological parameter, computer program products and processes associated with such devices.

### STATE OF THE ART

Devices for obtaining a physiological parameter are known. However these are not easy to use in all circumstances or are limited in the functions they propose and in their capacity to effectively help the user.

### PRESENTATION

An aim of the invention is to eliminate at least one of the disadvantages presented above.

For this purpose, a device is provided for obtaining at least one physiological parameter, for example of an individual, the device preferably being a portable device, comprising:
body temperature measuring means, for example of the individual,
display means configured to display the physiological parameter, the physiological parameter comprising the body temperature,
communication means, for example with a communications network to which equipment is connected, in particular wireless, and
data-processing means configured to transmit the physiological parameter to the equipment via the network.

The invention is advantageously completed by the following characteristics, taken alone or in any of their technically possible combinations:
the device also comprises means of receiving and processing data for measuring oxygen-pulsed saturation, for example of the individual;
the device also comprises means of receiving and processing data for measuring arterial pressure, for example of the individual;
the device also comprises means of receiving and processing data for measuring electrical heart activity, for example of the individual;
the device also comprises means of receiving and processing data for measuring glycaemia, for example of the individual;
the device also comprises a casing, at least partially housing the temperature measuring means, and/or the means of receiving and processing data for measuring oxygen-pulsed saturation, and/or the means of receiving and processing data for measuring arterial pressure, and/or the means of receiving and processing data for measuring electrical heart activity, and/or the means of receiving and processing data for measuring glycaemia;
a motor and/or a pump of an arterial pressure sensor housed at least partially inside the casing;
the pump comprises a buffer chamber adapted to limit the noise of the pump when operating;
the casing has a length less than or equal to 170 cm and/or a width less than or equal to 170 cm, for example 120 cm, for example 100 cm, for example 80 cm, and/or a thickness less than or equal to 170 cm, for example 120 cm, for example 100 cm, for example 80 cm, for example 50 cm, for example 25 cm;
the temperature measuring means are for example thermally insulated, preferably by means of a thermally insulating wall, from at least one part of the communication means and/or the processing means;
the thermally insulating wall extends for example between a first part and a second part of the casing, the temperature measuring means being arranged in the region of the first part;
the device has a mass less than or equal to 5 kg, for example 2 kg, for example 1 kg, for example 500 g.

An assembly comprising such device is also provided.

The invention is advantageously completed by the following characteristics, taken alone or in any of their technically possible combinations:
a connector;
the connector is a connector with an electric heart activity sensor, the device being configured such that when the device is physically connected to the electric heart activity sensor via the connector, a battery of the device cannot be charging and/or the device cannot be supplied by an external power supply;
the connector comprises a first part complementary to a port arranged terminating and/or arranged in the region of an opening of the casing;
the connector comprises a second blocking part, such that when the first part is physically connected to said port arranged terminating and/or arranged in the region of the opening of the casing, the second blocking part is arranged so as to prevent any connection to a connection port means having charging means of the battery and/or connection means to the external power supply.

The invention also relates to a method for obtaining physiological parameters executed by means of the device or the assembly such as described hereinabove. The invention also relates to a memory or a computer program product comprising instructions for executing this method.

The invention also relates to a method of use of physiological parameters which are measurable, measured and/or provided or which can be provided, executed by means of the device such as described hereinabove. The invention also relates to a memory or a computer program product comprising instructions for executing this process.

### DRAWINGS

Other aims, characteristics and advantages will emerge from the following description given by way of illustration and non-limiting in reference to the drawings, in which:

FIG. 1 shows a perspective view of a device according to an example of an embodiment of the invention;

FIG. 2 shows a method for obtaining physiological parameters according to an example of an embodiment of the invention;

FIGS. 3a to 3f show display images of the device of FIG. 1 during execution of the method of FIG. 2 according to an example of an embodiment of the invention;

FIG. 4 shows a method of use of physiological parameters according to an example of an embodiment of the invention;

FIGS. 5a to 5f show display images of the device of FIG. 1 during execution of the method of FIG. 4 according to an example of an embodiment of the invention;

FIG. 6a shows a perspective view of a device according to an example of an embodiment of the invention;

FIG. 6b shows a view of a left part of the device of FIG. 6a;

FIG. 6c shows an assembly comprising the device of FIG. 6a and a connector with an electric heart activity sensor.

### DESCRIPTION

### General Structure of the Device

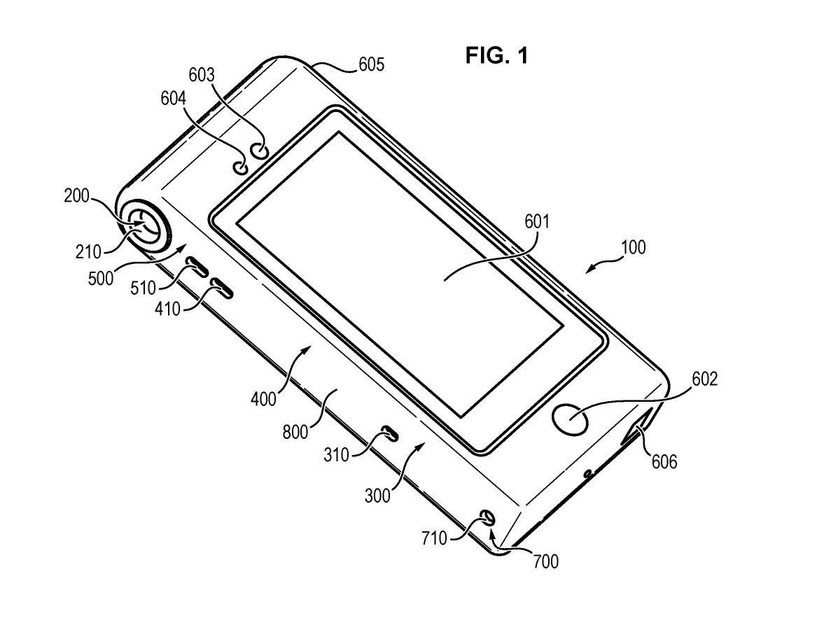

In reference to FIG. 1, a device for obtaining at least one physiological parameter, for example measurement of at least one physiological parameter, for example of an individual, for example of an animal, for example of a mammalian, for example of a human being is described.

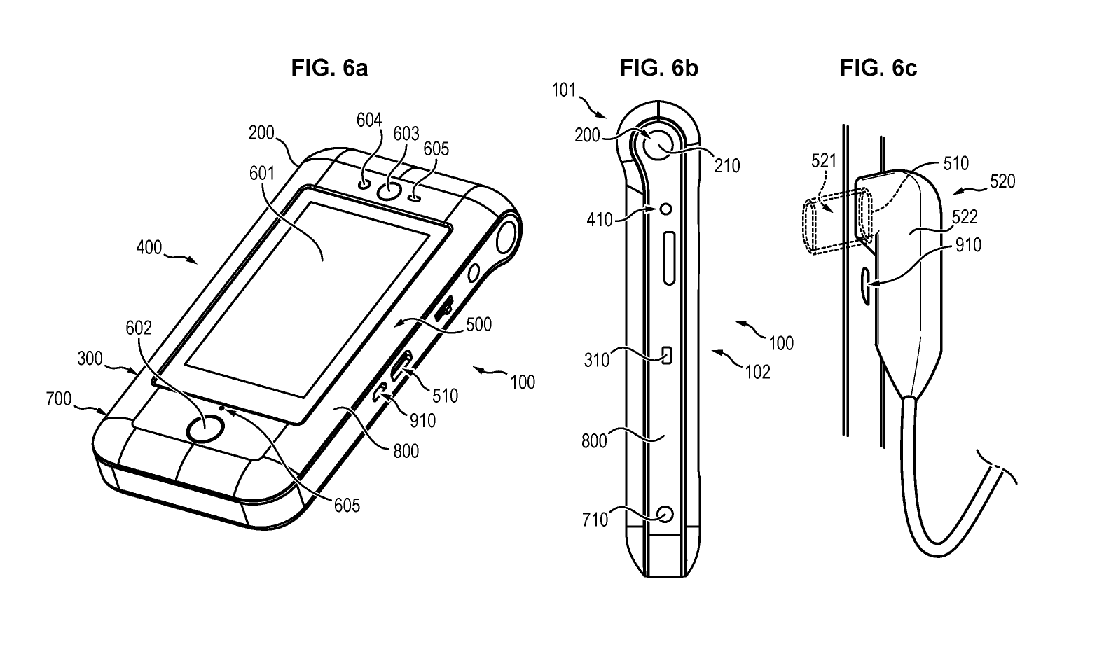

Similarly, in reference to FIGS. 6a, 6b and 6c, a device for obtaining at least one physiological parameter, for example measurement of at least one physiological parameter, for example of an individual, for example of an animal, for example of a mammalian, for example of a human being is described.

The device is preferably a portable device, that is, it has limited weight and bulk so it can be transported by an individual. The device for example has a mass less than or equal to 5 kg, for example 2 kg, for example 1 kg, for example 500 g.

The device comprises for example a casing 100. The casing 100 is for example adapted to contain or house one or more parts of the device, for example so as to protect said part or parts.

The casing has for example dimensions, for example a length and/or a width and/or a thickness, less than or equal to 30 cm, for example 20 cm. The casing has for example a length less than or equal to 170 cm. The casing has for example a width less than or equal to 170 cm, for example 120 cm, for example 100 cm, for example 80 cm. The casing has for example a thickness less than or equal to 170 cm, for example 120 cm, for example 100 cm, for example 80 cm, for example 50 cm, for example 25 cm. Accordingly it is possible to produce a portable device easily transportable, for example easily transportable in a small suitcase or a pocket.

The device is for example autonomous, that is, it comprises all the elements needed to operate, power supply for example. It therefore allows nomadic operation.

### Communication Means

The device comprises for example communication means 800 with a communications network, for example by means of a local connection and/or a connection with a remote server. The communication is wireless communication, for example. The communication is for example executed by means of a protocol of Wi-Fi and/or Bluetooth and/or GPS and/or 2G and/or 3G and/or LTE type. Un equipment is for example connected to the communications network. The equipment can be a fixed or mobile terminal.

The device can comprise or form a terminal mobile, for example a telephone, for example apparatus of “smartphone” type.

### Data Processing

The device can comprise data-processing means. The data-processing means are for example configured to transmit the physiological parameter to the equipment via the network, for example at least one physiological parameter measured or provided by the device, for example via the means 200, 300, 400, 500 and/or 700 such as described hereinbelow.

The data-processing means comprise for example the processing means data such as described hereinbelow, such as the means 200, 300, 400, 500, and/or 700.

The device can also comprise other data-processing means.

The data-processing means can for example be executed by at least one processor, for example a central processing unit.

The device can also comprise at least one graphic processor.

The device can comprise data-storage means, for example of read only memory and/or random access memory type.

The device can comprise an operating system.

The device can comprise multimedia processing means, for example in audio and/or video and/or image format, for example such reading and/or display means.

The device can comprise a battery. The device can comprise charging means of the battery and/or connection means to charging means of the battery, for example a port. The device can comprise a light indicator of the status of the battery.

The device can comprise means for insertion of a memory card and/or security card, for example a SIM card.

The port of the connection means to the charging means of the battery is for example arranged terminating and/or arranged in the region of at least one opening 910 of the casing 100.

The device can comprise connection means to an external power supply, for example a connection port to an external power supply. The port of the connection means to an external power supply is for example arranged terminating and/or arranged in the region of at least one opening 910 of the casing 100. The port of the connection means to charging means of the battery forms for example the connection port to an external power supply.

### Interface

The device can comprise an interface, for example a user interface. The interface can comprise data input and/or output means.

The device can comprise display means 601. The display means 601 comprise for example at least one screen, for example a screen of LCD type, for example a touch screen. The display means are for example arranged in the region of a front face of the casing 100, for example so as to define relative to the front face a rear face opposite to the front face and one or more lateral faces connecting the front face and the rear face. The display means 601 are for example configured to display information legible according to at least one given orientation, so as to define according to one of these orientations an upper part, a lower part, a left part and a right part of the front face and/or of the casing 100.

The display means 601 are for example configured to display the at least one physiological parameter, for example at least one physiological parameter measured or provided by the device, for example by the means 200, 300, 400, 500 and/or 700 such as described hereinbelow.

The device can comprise tactile input means for example comprising at least one tactile sensor 602. The device can comprise a touch screen which can be separate or combined with the display means 601. The device can comprise one or more buttons and/or keys and/or keypads.

The device can comprise at least one camera and/or photo unit 603, for example accompanied by a flash 604.

The device can comprise a loudspeaker and/or an earphone 605 and/or a microphone 606.

The device can comprise a gyroscopic sensor and/or an accelerometer and/or a light sensor and/or a proximity sensor and/or a Hall effect sensor.

The device can comprise physical connection means to at least one sound output, for example a port of Jack type.

The data-processing means and the interface are for example configured to allow the control of the measuring of one or more physiological parameters, for example of those described hereinbelow and/or the consultation of the value or values measured in real time. The device is for example configured for storing the data measured, for example in the form of history and to allow them to be consulted.

The device can be configured to compare the measured value or values, for example in real time, with at least one reference value, and provide an estimation of incurred risk and/or an initiation of a connection via the connection means, so as to put the user of the device in communication with a computer server to help him (her) and/or with a remote participant. For example, if a value measured is greater or less than a predetermined reference value, for example if the arterial pressure is above a certain threshold, the device can automatically set a remote connection with a participant, for example a person of a support service and/or a specialist in the medical field. For example, if a value measured is greater or less than a predetermined reference value, for example if the arterial pressure is greater than a certain threshold, the device can provide a light indicator of a colour dependent on comparison, the light indicator being an indicator of a level of risk for the health of the user. The light indicator is for example a symbol and/or a colour.

### Temperature

The device comprises for example temperature measuring means 200, for example a temperature as physiological parameter, for example a body temperature, for example a body temperature of the individual, for example of the body of an animal, for example of the body of a mammalian, for example of the human body. The temperature measuring means 200 are adapted for example to provide the measured temperature.

The temperature measuring means 200 are for example such that the device forms a thermometer.

The temperature measuring means 200 are for example contactless temperature measuring means, for example via infrared.

The temperature measuring means 200 comprise for example a temperature sensor, for example a sensor of infrared type.

The temperature measuring means 200 are for example adapted to measure a temperature in a range of given values, for example a temperature between 0 and 100° C., for example between 5 and 50° C., for example greater than 5° C., for example between 20 and 50° C., for example less than 45° C., for example between 30 and 40° C.

The temperature measuring means 200 are for example adapted to measure a temperature with a precision of the order of Celsius degree, for example of the order of Celsius semi-degree, for example of the order of a fifth of Celsius degree, for example of the order of a tenth of Celsius degree.

The temperature measuring means 200 are for example arranged in the region of the casing 100, for example in the casing 100, for example incorporated into the casing 100, for example attached to the casing 100. The temperature measuring means 200 are housed for example at least partially inside the casing 100, for example extending at least in part inside the casing 100. The temperature measuring means 200 extend for example in an end part of the casing, for example in the region of the upper part, for example of the upper part arranged above the display means 601, for example in a direction defined by left and right, for example according to a width and/or a length of the casing 100 and/or of the front face.

The casing 100 comprises at least one opening 210 in the region of which the temperature measuring means 200 terminate, for example the temperature sensor of the temperature measuring means 200, so as to allow measuring of temperature, for example remotely, for example by orientation of the opening 210 in the direction of a zone to be measured, for example of the temperature of an individual. The opening 210 is for example made in the region of a lateral face of the casing 100.

The temperature measuring means 200 allow to obtain a measurement of the temperature easily by means of the device, the device which can process the information or transmit it and supply it to a user of the device or to a remote server. Such a device can be used both by a health sector specialist such as a doctor or nurse and by an individual, for example the patient or a relative who is not a specialist in this field.

### Oxygen-Pulsed Saturation

The device can comprise means of receiving and processing data for measuring oxygen-pulsed saturation 300, for example originating from measuring oxygen-pulsed saturation, in particular performed by an oxygen-pulsed saturation sensor connected to the device. Oxygen-pulsed saturation is for example that of the individual, for example of the body of an animal, for example of the body of a mammalian, for example of the human body. The measurement of oxygen-pulsed saturation is for example of absorption spectrometry type. The means of receiving and processing data for measuring oxygen-pulsed saturation 300 are for example adapted to provide the measured oxygen-pulsed saturation.

Such a measurement allows to especially quantify the oxygen saturation of the haemoglobin in the region of the blood capillaries of the individual.

For this purpose, the device can comprise connection means, for example physical connection means, to an oxygen-pulsed saturation sensor, comprising for example a port arranged terminating and/or arranged in the region of an opening 310 of the casing 100. The connection means are for example adapted to send and/or receive data to/from the oxygen-pulsed saturation sensor.

The means of receiving and processing data for measuring oxygen-pulsed saturation 300 comprise for example control means of the oxygen-pulsed saturation sensor.

The oxygen-pulsed saturation sensor is for example of finger cot and/or self-adhesive type, for example infrared. The oxygen-pulsed saturation sensor comprises for example a probe. The pulsed saturation sensor comprises for example a light source adapted to emit in two distinct wavelengths, for example one wavelength in red, for example one wavelength in infrared. The light source comprises for example at least one LED transmitter, for example a plurality of LED transmitters, one for each wavelength. The sensor comprises for example a light receiver, for example of photodiode type, adapted to receive a signal coming from the light source, the light receiver being for example a high-sensitivity receiver.

The device can comprise such an oxygen-pulsed saturation sensor (not shown), with the means of receiving and processing data for measuring oxygen-pulsed saturation 300 forming measuring means of oxygen-pulsed saturation.

The means of receiving and processing data for measuring oxygen-pulsed saturation 300 are for example adapted to process and/or perform oxygen saturation measurements in a range of given values, for example saturation between 0 and 100%, for example between 25 and 100%, for example between 50 and 100%. The means of receiving and processing data for measuring oxygen-pulsed saturation 300 are for example adapted to process and/or perform oxygen saturation measurements when the pulse is between 0 and 300 bpm, for example between 25 and 250 bpm, for example between 25 and 200 bpm.

The means of receiving and processing data for measuring oxygen-pulsed saturation 300 are for example adapted to process and/or perform oxygen saturation measurements with a precision of the order of 10%, for example of the order of 7%, for example of the order of 5%, for example of the order of 4%, for example of the order of 4% at the neonatal stage, for example of the order of 4% in motion, for example of the order of 3% at rest, in particular when the saturation measured is between 70% and 100%.

The means of receiving and processing data for measuring oxygen-pulsed saturation 300 are for example arranged in the region of the casing 100, for example in the casing 100, for example incorporated into the casing 100, for example attached to the casing 100. The means of receiving and processing data for measuring oxygen-pulsed saturation 300 are housed for example at least partially inside the casing 100, and for example extend at least in part inside the casing 100.

The oxygen-pulsed saturation sensor can be arranged in the region of the casing 100, for example so as to be placed removably in the casing 100, for example incorporated into the casing 100, for example attached to the casing 100. The oxygen-pulsed saturation sensor is housed for example at least partially inside the casing 100, and for example extends at least partially inside the casing 100.

The means of receiving and processing data for measuring oxygen-pulsed saturation 300 for receiving and/or obtaining a measurement of the oxygen saturation easily by means of the device, the device which can process the information or transmit it and provide it to a user of the device or to a remote server. Such a device can be used both by a health sector specialist such as a doctor or a nurse and by an individual, for example the patient or a relative who is not a specialist in this field.

In addition to oxygen-pulsed saturation, the means of receiving and processing data for measuring oxygen-pulsed saturation 300 can be adapted to provide a pulse, for example a pulse of between 25 and 250 bpm, for example with a precision of the order of 5%.

### Arterial Pressure

The device can comprise means of receiving and processing data for measuring arterial pressure 400, coming from measurement of arterial pressure, in particular carried out by an arterial pressure sensor connected to the device. The arterial pressure is for example that of the individual, for example of the body of an animal, for example of the body of a mammalian, for example of the human body. Measuring the arterial pressure is for example of the arm type. The means of receiving and processing data for measuring arterial pressure 400 are for example adapted to provide the measured arterial pressure. The arterial measured pressure comprises for example the systolic and/or diastolic, and/or average arterial pressure.

For this purpose, the device can comprise connection means, for example physical connection means, to an arterial pressure sensor, comprising for example a port arranged terminating and/or arranged in the region of at least one opening 410 of the casing 100. The connection means are for example adapted to send and/or receive data to/from the arterial pressure sensor.

The means of receiving and processing data for measuring arterial pressure 400 comprise for example control means of the arterial pressure sensor.

The arterial pressure sensor is for example of cuff type. The arterial pressure sensor comprises for example an inflatable cuff, and/or a motor actuating a pump adapted to inflate/deflate the inflatable cuff. The arterial pressure sensor comprises for example a strain gauge with semi-conductor and/or a pressure transducer.

The device can comprise such an arterial pressure sensor (not shown) forming arterial pressure measuring means with the means of receiving and processing data for measuring arterial pressure 400.

The means of receiving and processing data for measuring arterial pressure 400 are for example adapted to process and/or take measurements of arterial pressure in a range of given values, for example an arterial pressure between 0 and 400 mmHg, for example between 0 and 300 mmHg, for example between 0 and 250 mmHg. The means of receiving and processing data for measuring arterial pressure 400 are for example adapted to process and/or take arterial pressure measurements when the pulse is between 0 and 300 bpm, for example between 25 and 250 bpm, for example between 25 and 200 bpm, for example between 40 and 180 bpm.

The means of receiving and processing data for measuring arterial pressure 400 are for example adapted to process and/or take arterial pressure measurements with a precision of the order of 10%, for example of the order of 7%, for example of the order of 5%, for example of the order of 4%.

The means of receiving and processing data for measuring arterial pressure 400 are for example arranged in the region of the casing 100, for example in the casing 100, for example incorporated into the casing 100, for example fixed to the casing 100. The arterial pressure sensor is housed for example at least partially inside the casing 100, and for example extends at least in part inside the casing 100, the motor and/or the pump of the arterial pressure sensor being housed for example at least partially inside the casing 100, for example extending at least in part inside the casing 100, for example in an end part of the casing 100, for example in the region of the lower part, for example of the lower part arranged below the display means 601, for example in a direction defined by the left and the right, for example according to a width and/or a length of the casing 100 and/or of the front face.

The means of receiving and processing data for measuring arterial pressure 400 for receiving and/or obtaining a measurement of the arterial pressure easily by means of the device, the device which can process the information or transmit it, and provide it to a user of the device or to a remote server. Such a device can be used both by a health sector specialist such as a doctor or a nurse, and by an individual, for example the patient or a relative who is not a specialist in this field.

In addition to the arterial pressure, the means of receiving and processing data for measuring arterial pressure 400 can be adapted to provide a pulse, for example a pulse of between 40 and 180 bpm, for example with a precision of the order of 5%.

### Electrical Heart Activity

The device can comprise means of receiving and processing data for measuring electrical heart activity 500, for example coming from measurement of electrical heart activity, in particular performed by a sensor electrical heart activity connected to the device. The electrical heart activity is for example that of the heart the individual, for example of the heart of an animal, for example of the heart of a mammalian, for example of the human heart. The measurement of electrical heart activity is for example of electrocardiograph type. The means of receiving and processing data for measuring electrical heart activity 500 are for example adapted to provide the measured electrical heart activity. The measured electrical heart activity comprises for example electrocardiography, for example of type ECG at 5 leads and/or ECG at 12 leads.

For this purpose, the device can comprise connection means, for example physical connection means, with an electric heart activity sensor, comprising for example a port arranged terminating and/or arranged in the region of at least one opening 510 of the casing 100. The connection means are for example adapted to send and/or receive data to the electric heart activity sensor.

The means of receiving and processing data for measuring electrical activity 500 comprise for example control means of the arterial pressure sensor.

The sensor electrical heart activity is for example of 5 leads and/or 12 leads type. The arterial pressure sensor comprises for example electrodes and leads.

The device can comprise such a electrical heart activity sensor (not shown) forming measuring means electrical heart activity, for example an electrocardioscope with the means of receiving and processing data for measuring electrical heart activity 500.

The means of receiving and processing data for measuring electrical heart activity 500 are for example adapted to process and/or take measurements of electrical heart activity in a range of given values, for example an offset voltage of direct current (“DC offset voltage” in English terminology) of between −500 and +500 mV, for example between −300 and +300 mV, for example a differential voltage measurement (“Differential voltage measurement” in English terminology) of the order of 5 mV.

The means of receiving and processing data for measuring electrical heart activity 500 are for example adapted to process and/or take measurements of electrical heart activity with a precision of the order of 10%, for example of the order of 5%, for example of the order of 2%, for example of the order of 1%.

The means of receiving and processing data for measuring electrical heart activity 500 are for example arranged in the region of the casing 100, for example in the casing 100, for example incorporated into the casing 100, for example attached to the casing 100. The sensor electrical heart activity is housed for example at least partially inside the casing 100, for example extends at least in part inside the casing 100.

The means of receiving and processing data for measuring electrical heart activity 500 for receiving and/or obtaining a measurement electrical heart activity easily by means of the device, the device which can process the information or transmit it and provide it to a user of the device or to a remote server. Such a device can therefore be used both by a health sector specialist such as a doctor or a nurse, and by an individual, for example the patient or a relative who is not a specialist in this field.

### Glycaemia

The device can comprise means of receiving and processing data for measuring glycaemia 700, for example coming from measurement of glycaemia 700, in particular performed by a glycaemia sensor connected to the device. The glycaemia is for example that of the individual, for example of the body of an animal, for example of the body of a mammalian, for example of the human body. The means of receiving and processing data for measuring glycaemia 700 are for example adapted to provide the measured glycaemia.

For this purpose, the device can comprise connection means, for example physical connection means, to a glycaemia sensor, comprising for example a port arranged terminating and/or arranged in the region of at least one opening 710 of the casing 100. The connection means are for example adapted to send and/or receive data to/from the glycaemia sensor.

The means of receiving and processing data for measuring glycaemia 700 comprise for example glycaemia control means.

The device can comprise such a glycaemia sensor (not shown) forming glycaemia measuring means, for example a glycometer with the means of receiving and processing data for measuring glycaemia 700.

The means of receiving and processing data for measuring glycaemia 700 are for example arranged in the region of the casing 100, for example in the casing 100, for example incorporated into the casing 100, for example attached to the casing 100. The glycaemia sensor is housed for example at least partially inside the casing 100, for example extends at least in part inside the casing 100.

The means of receiving and processing data for measuring glycaemia 700 for receiving and/or obtaining a glycaemia measurement easily by means of the device, the device which can process the information or transmit it and provide it to a user of the device or to a remote server. Such a device can be used both by a health sector specialist such as a doctor or a nurse, and by an individual, for example the patient or a relative who is not a specialist in this field.

### Casing

The means 200, 300, 400, 500 and/or 700 are for example thermally insulated, for example by means of a wall 201, from one or more of the remaining means 200, 300, 400, 500 and/or 700 and/or communication means and/or data-processing means, and/or from at least one other heat-producing element arranged at least partially in the casing 100. Accordingly it is possible to limit or prevent disruption to the measurement.

The temperature measuring means 200 are for example thermally insulated, for example by means of a thermally insulating wall 201, from at least one part of the communication means and/or the data-processing means, for example from at least one chip, for example a chip comprising a microcontroller and/or a memory, for example a SIM card. The temperature measuring means 200 are for example arranged in the region of a first part 101 of the casing 100, for example the upper part. The communication means, for example the SIM card, extend for example at least partially, for example entirely, for example in the region of a second part 102 of the casing 100, for example the lower part. The thermally insulating wall 201 extends for example between the first part 101 and the second part 102. It is possible to ensure proper taking of temperature, in particular contactless, while limiting the size of the device and/or its components.

The pump of the arterial pressure sensor comprises for example a buffer chamber. The buffer chamber forms for example an air filter. The buffer chamber is for example adapted to limit the noise of the pump when operating. This is particularly useful in the case of miniaturization of the pump, the miniaturization needing structural adaptations which can cause noises to occur. It is therefore possible to ensure operation with reduced noise and limit the size of the device and/or its components.

The pump is for example arranged in the region of the second part. It is possible to optimise the structure of the device to provide an optimised device of reduced size and reliable.

In reference to FIG. 6c, the assembly comprising such a device also comprises for example a connector 520, for example with an electric heart activity sensor, comprising a first part 521 complementary to the port arranged terminating and/or arranged in the region of the opening 510 of the casing 100, which is for example a female port. The first part is for example a plug, for example a standardised male plug (in particular as per the USB standard, but other standards such as RJ45 or IEEE1394 are possible) adapted to engage in a compatible female port. The connector 520 is adapted to send and/or receive data between the sensor electrical heart activity and the port arranged terminating and/or arranged in the region of the opening 510 of the casing 100, for example when the first part 521 complementary to the port is connected physically to said port, for example attached to said port, for example placed in the region of said port, for example in said port. The connector 520 is for example extended by a connection cable to the electric heart activity sensor and/or directly to the electric heart activity sensor.

The device is for example configured such that, when the device is connected physically to the electric heart activity sensor, in particular when the connector 520 is attached to said port arranged terminating and/or arranged in the region of the opening 510 of the casing 100, the battery cannot be charging and/or the device cannot be supplied by an external power supply. For example, in these conditions, the charging means cannot be connected to the connection means with charging means of the battery, for example the port. For example, in these conditions, the external feed cannot be connected to the connection means to an external feed, for example the port.

It is therefore possible to avoid having to dimension the device to ensure simultaneously both electrical activity measurement and charging of the battery or supply. It is possible to make a device of reduced size.

It is also possible to prevent substantial heating due to use of both means and therefore all the more risking impairing temperature measurement or needing thermal insulation involving a larger volume of the device.

For example, the connector 520 can comprise a second blocking part 522, such that when the first part 521 complementary to the port is connected physically to said port arranged terminating and/or arranged in the region of the opening 510 of the casing 100, for example placed in the region of said port, for example in said port, the second blocking part 522 is arranged so as to prevent any connection to the port of the connection means with charging means of the battery and/or connection means to an external power supply. The second blocking part 522 is for example arranged to block access to the port arranged terminating and/or arranged in the region of the opening 910 of the casing 100, for example by being placed immediately in front of said opening 910 or in front of said port. The second part 522 is for example bent so as to allow such a blocking.

In the same way, the device is for example configured such that, when the device is connected physically via another port of the device, the battery cannot be charging and/or the device cannot be supplied by an external power supply.

The physical connection means with an electric heart activity sensor, the connection means with charging means of the battery and/or the connection means to an external power supply are for example arranged in the region of the second part 102 of the casing 100. It is therefore possible to further optimize the configuration of the device to provide a functional device of reduced size.

Alternatively or in addition, the assembly comprising such a device also comprises for example a connector, for example an external power supply, comprising for example a first part complementary to the port arranged terminating and/or arranged in the region of the opening 910 of the casing 100, which is for example a female port. The first part is for example a plug, for example a standardised male plug (in particular as per the USB standard, but other standards such as RJ45 or IEEE1394 are possible) adapted to engage in a compatible female port. Said connector is adapted to electrically supply the battery and/or the device from charging means and/or an external power supply, for example when the first part complementary to the port is connected physically to said port, for example placed in the region of said port, for example in said port. The connector is for example extended by a connection cable to the charging means and/or to the external power supply.

The device is for example configured such that, when the device is connected physically to the charging means of the battery and/or to the external power supply, in particular when the connector is attached to said port arranged terminating and/or arranged in the region of the opening 910 of the casing 100, the electrical heart activity sensor does not or cannot function. For example, in these conditions, the plug 520 cannot be connected to the port arranged terminating and/or arranged in the region of the opening 510 of the casing 100.

It is therefore possible to avoid having to dimension the device to ensure simultaneously both electrical activity measurement and charging of the battery or supply. It is possible to make a device of reduced size

Furthermore, it is possible to prevent substantial heating due to use of both means and therefore all the more risking impairing temperature measurement or needing thermal insulation involving a larger volume of the device

For example, the plug can comprise a second blocking part, such that when the first part complementary to the port is connected physically to said port arranged terminating and/or arranged in the region of the opening 910 of the casing 100, for example placed in the region of said port, for example in said port, the second blocking part is arranged so as to prevent any connection to the port arranged terminating and/or arranged in the region of the opening 510 of the casing 100, for example by being placed immediately in front of said opening 510 or in front of said port. The second part is for example bent so as to allow such a blocking.

In the same way, the device is for example configured such that, when the device is connected physically to the charging means of the battery and/or to the external power supply, a dedicated plug cannot be connected to one of the other ports of the device.

### Examples of Embodiments of the Device

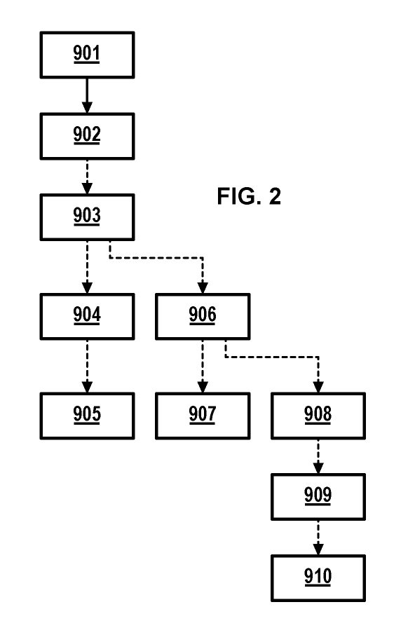

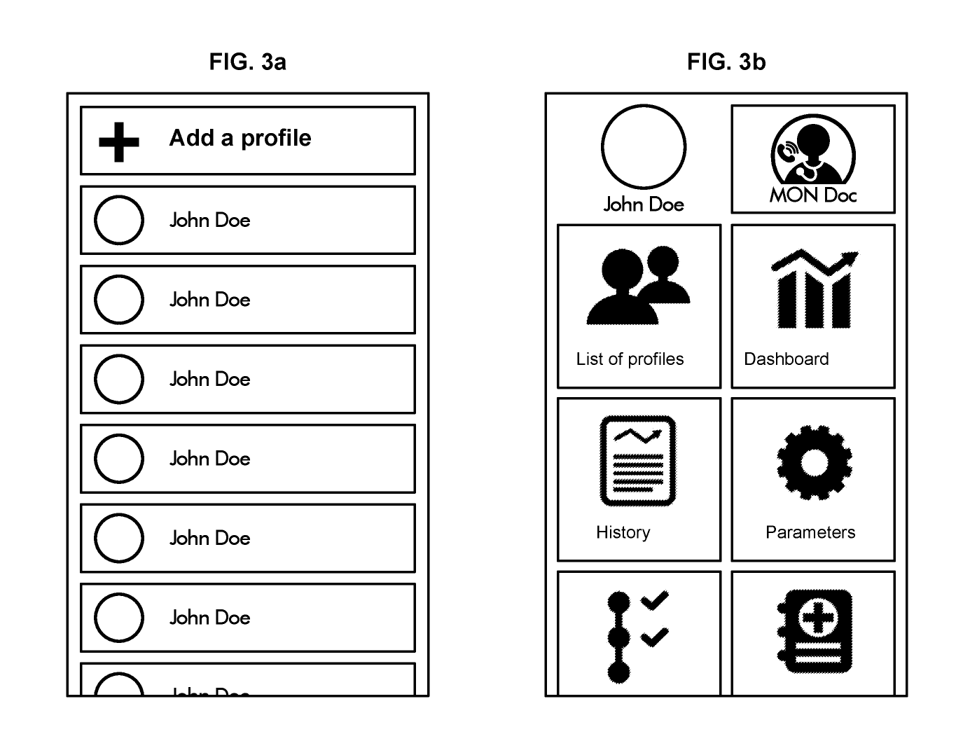

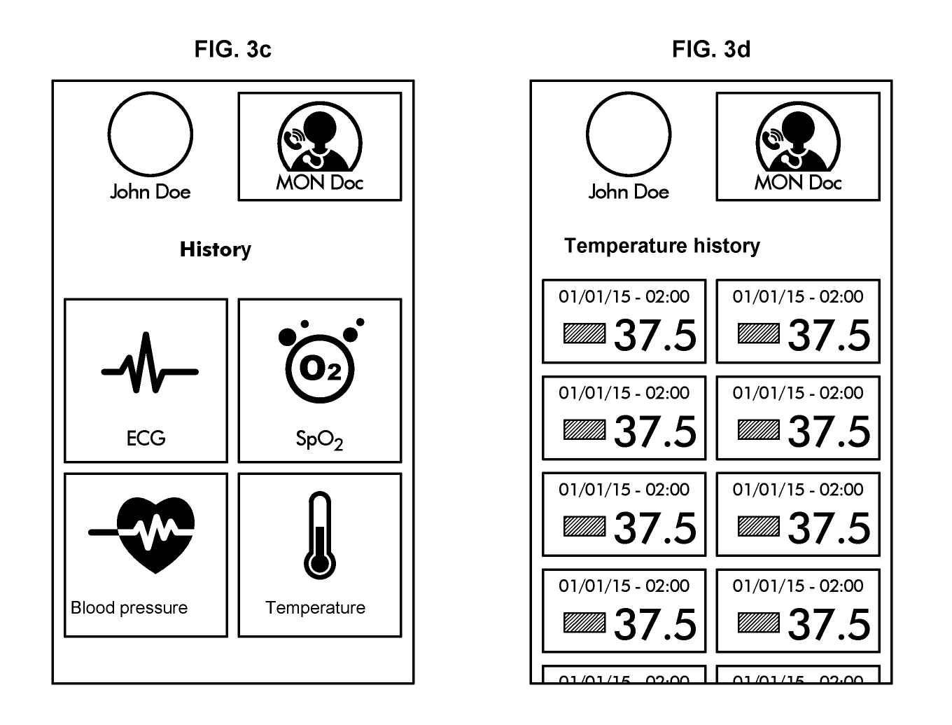

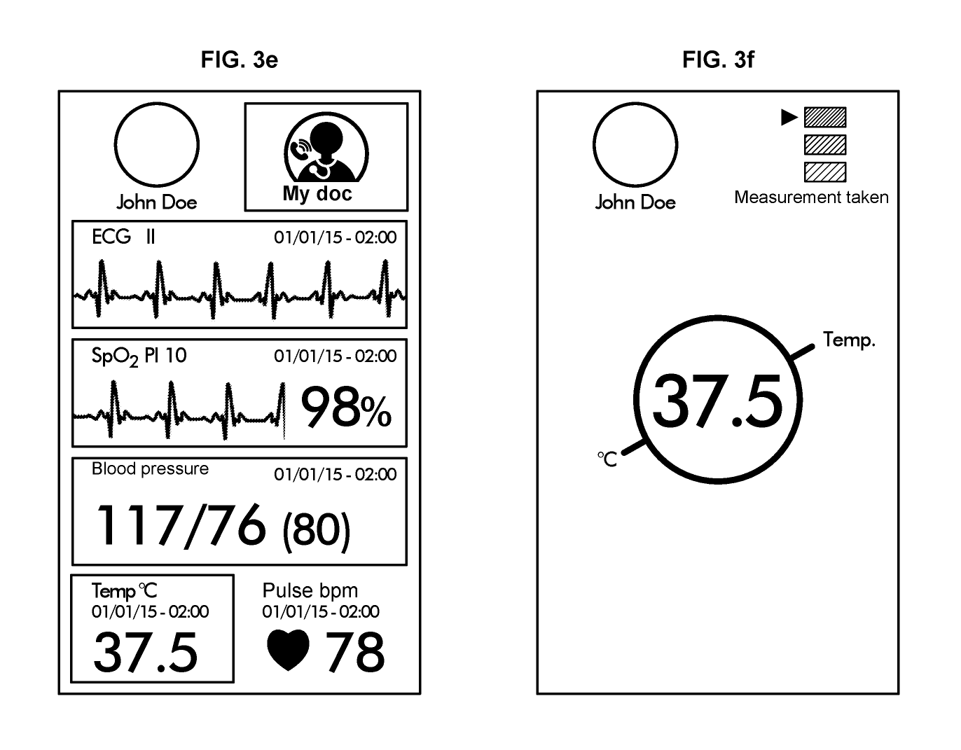

In reference to FIG. 2 and to FIGS. 3a to 3f, a method for obtaining physiological parameters executed by means of the device such as described hereinabove is described. The device can comprise a memory on which the instructions corresponding to this method are stored.

A computer program product comprising instructions for executing the method can also be provided.

According to a first step 901, the device can display a list including a plurality of profiles corresponding to different users of the device, for example in the region of the display means 601. The step 901 is for example illustrated by FIG. 3a.

According to a second step 902, in response to a user input, for example by means of the user interface, a profile can be selected from the plurality of profiles. According to a third step 903, a user profile can be displayed, for example following selection of the profile during the step 902. The step 903 is for example illustrated by FIG. 3b.

According to a fourth step 904, in response to a user input, for example by means of the user interface, for example in the region of the user profile displayed in the step 903, a list of physiological parameters including the history is available and can be displayed. The step 904 is for example illustrated by FIG. 3c. According to a fifth step 905, in response to a user input, for example by means of the user interface, for example from the list of physiological parameters displayed in the step 904, the history of a physiological parameter can be selected and displayed. It is possible to easily consult the history of several measurements performed by means of the device. The step 905 is for example illustrated by FIG. 3d.

According to a sixth step 906, in response to a user input, for example by means of the user interface, for example in the region of the profile user displayed in the step 903, a list of physiological parameters measurable by means of the device and/or which can be provided by the device can be displayed. The list of physiological parameters can display, for each physiological parameter, its measurement performed in real time and/or its final measurement performed and/or provided by the device. The step 906 is for example illustrated by FIG. 3e.

At the same time as the list of measurable physiological parameters the display means can display, for example during the sixth step 906, a symbol indicating the possibility of being put in contact with a remote server and/or holding a telephone conversation for communicating with a support service, for example with a health sector specialist.

According to a seventh step 907, in response to a user input, for example in the region of the symbol indicating the possibility of being put in contact, or automatically as a function of the result of comparison of at least one value of physiological parameter from the list of measurable physiological parameters and/or which can be provided by the device with at least one reference value. In this way, if a physiological parameter or a combination is set up as having a value or values potentially at risk, a support service or a health sector specialist can be contacted automatically.

According to an eighth step 908, in response to a user input, for example by means of the user interface, for example from the list of measurable parameters or which can be provided displayed at the step 906, a specific physiological parameter can be selected and displayed. It is then possible to display the final measurement performed or provided of the physiological parameter and/or the measurement performed or provided in real time of the physiological parameter. It is accordingly possible to easily consult the detail of the measurement performed by means of the device.

According to a ninth step 909, in response to a user input, for example by means of the user interface, for example from the display provided at the step 908, the value of the physiological parameter can be updated. The updating can comprise the display of instructions to take the measurement. It is possible to remind the user of the conditions to be respected for taking a measurement of a given physiological parameter, for example as it relates to breathing, the position of the sensor or of the body, or even the respiratory behaviour to be adopted.

In the steps 908 or 909, a visual indicator can provide a qualitative indication of the value obtained. For example a colored indicator can indicate whether the value is considered as normal or as characteristic of risk. The steps 908 and 909 are for example illustrated by FIG. 3f.

According to a tenth step 910, in response to a user input, for example by means of the user interface and/or automatically, the value of the updated physiological parameter can be saved. It is then possible to constitute a history, in particular a history comprising only those measurements performed correctly.

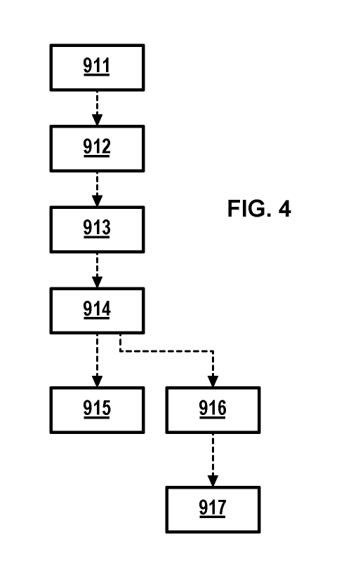

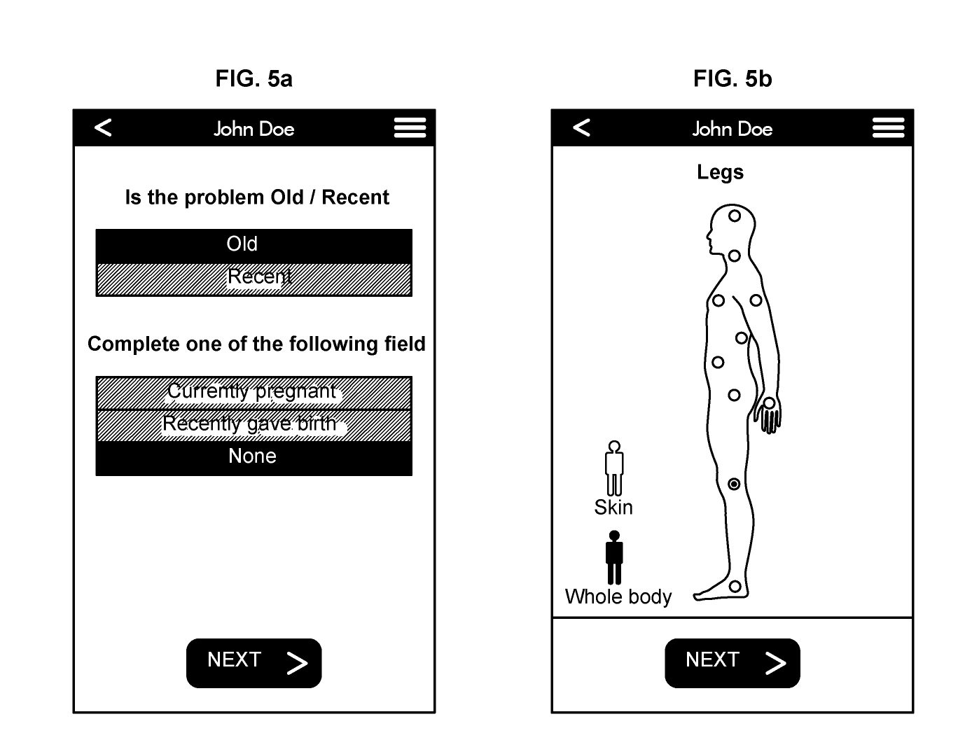

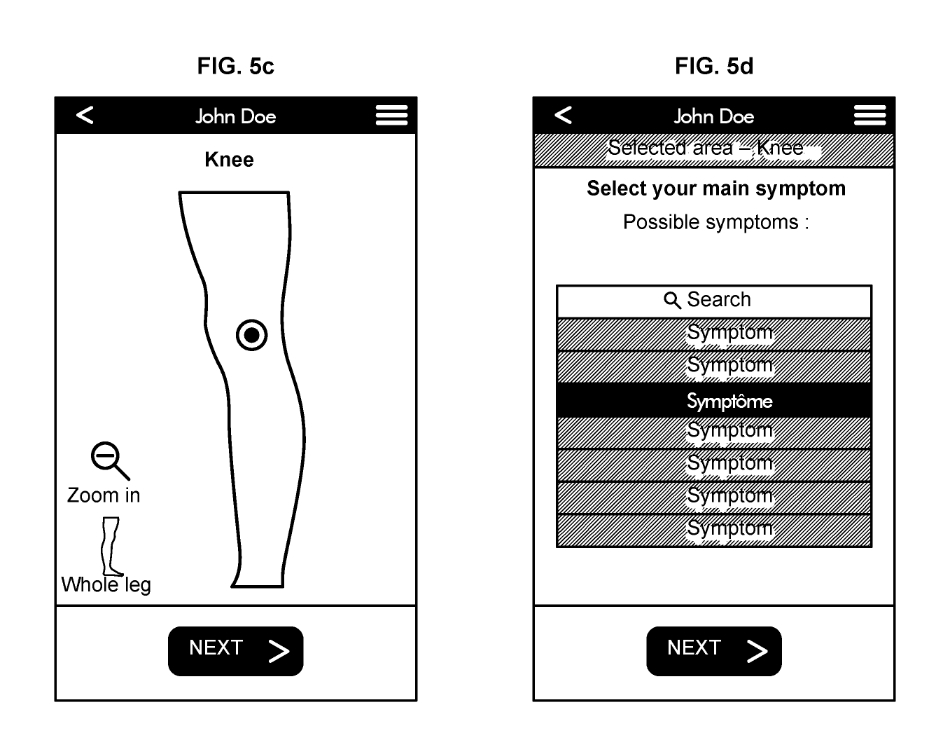

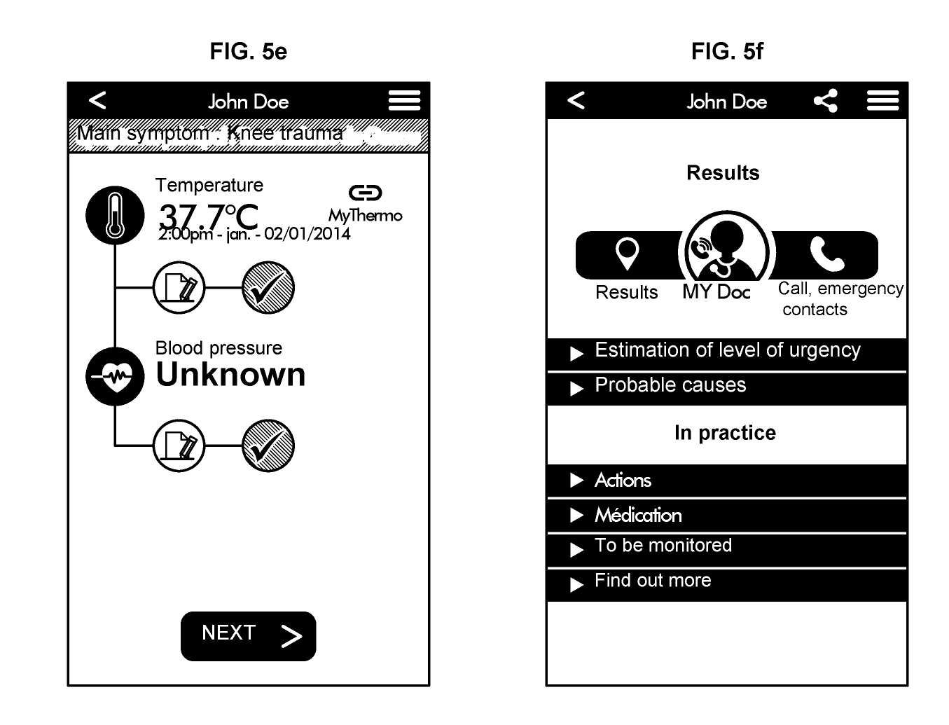

In reference to FIG. 4 and FIGS. 5a to 5f, a method of use of measurable physiological parameters, measured and/or provided or which can be provided, executed by means of the device such as described hereinabove is described. The device can comprise a memory on which the instructions corresponding to this method are stored.

A computer program product comprising instructions for executing the method can also be provided.

This method can be executed as part of the method for obtaining described hereinabove in reference to FIG. 2.

According to an eleventh step 911, the device can display, for example in the region of the display means 601, one or more information requests concerning the status of the user, for example if the reason for which he is here using the device is recent or old, for example if it concerns pain now being felt or present for several days, for example if the user is pregnant or not. The user can respond to these requests via one or more inputs in the region of the user interface. The step 911 is for example illustrated by FIG. 5a.

According to a twelfth step 912, for example in response to a user input, for example following the step 911, the device can display an information request concerning the zone of the body of the user where symptoms are being felt. In the region of the display means the device can in particular display a representation of the body of the user, the user which can display specific parts of the body to provide information on the precise area of the symptoms. The step 912 is for example illustrated by FIG. 5b. According to a thirteenth step 913, for example following the step 912, for example in response to a user input, in particular in the region of part of the display means 601 corresponding to a specific zone of the human body, a zone of the human body showing the symptoms to be analysed is selected. The step 913 is for example illustrated by FIG. 5c.

The steps 912 and 913 can be repeated to select a plurality of zones of the human body.

According to a fourteenth step 914, the device can display, for example in the region of the display means 601, one or more additional information requests concerning the status of the user and specific to the zone or zones of the human body selected at the step 913. The step 914 is for example illustrated by FIG. 5d. According to a fifteenth step 915, the device can send a report to a remote server following the step 915. It is thereby possible to constitute a history of the symptoms presented by the user over time.

In a sixteenth step 916, at least one value of a physiological parameter is measured and/or provided by means of the device as described hereinabove, for example by means of the steps 908 and/or 909 and/or 910 described hereinabove.

The display means can display, for example during the sixteenth step 916 or when it is completed, a symbol indicating the possibility of being put in contact with a remote server and/or holding a telephone conversation to communicate with a support service, for example with a health sector specialist. The step 916 is for example illustrated by FIGS. 5e and 5f.

According to a seventeenth step 917, in response to a user input, for example in the region of the symbol indicating the possibility of being put in contact or automatically as a function of the result of the comparison of at least one value of a physiological parameter measured and/or provided by the device at the step 916 with at least one reference value. In this way, if a physiological parameter or a combination is set up as having a value or values potentially at risk, a support service or health sector specialist can be contacted automatically.

The method for obtaining physiological parameters and/or use of the measurable physiological parameters can comprise a step for measuring at least one of the physiological parameters. The measuring step can be conducted in response to a measurement instruction coming from the remote network. The instruction triggering the measurement can be provided by a health sector specialist such as a doctor or a nurse who is in contact with the user, for example via the device. This is particularly advantageous since the specialist can ensure that the necessary conditions are fulfilled so that the measurement is made correctly and ensure that the results obtained are correct or that the measurement is made completely safely.

The method for obtaining physiological parameters and/or the use of physiological measurable parameters can comprise a step for calibrating and/recalibrating. The calibrating and/or recalibrating step can be conducted in response to a measurement instruction coming from the remote network. The instruction triggering the calibrating and/or recalibrating can be provided by a health sector specialist such as a doctor or a nurse who is in contact with the user, for example via the device. This is particularly advantageous since the specialist can ensure that the necessary conditions are fulfilled so that the measurement is taken correctly and ensure that the results obtained are correct.

The calibrating and/or the recalibrating is for example calibrating and/or recalibrating of the temperature measuring means 200. The calibrating and/or the recalibrating can comprise adaptation of the temperature relative to the ambient temperature. This is particularly advantageous since it is possible to reduce the size of the device by limiting the impact which the ambient temperature can have, immediately outside the device or around the temperature measuring means 200, on the reliability of the measurement.

1. Device for obtaining at least one physiological parameter of an individual, the device being a portable device comprising:
body temperature measuring means of the individual,
display means configured to display the physiological parameter, the physiological parameter comprising the body temperature,
communication means with a communications network to which equipment is connected, in particular wireless, and
processing means configured to transmit the physiological parameter to said equipment via the network.

2. The device according to claim 1, wherein the device also comprises means of receiving and processing data for measuring oxygen-pulsed saturation of the individual.

3. device according to claim 1, wherein the device also comprises means of receiving and processing data for measuring arterial pressure of the individual.

4. The device according to claim 1, wherein the device also comprises means of receiving and processing data for measuring electrical heart activity of the individual.

5. The device according to claim 1, wherein the device also comprises means of receiving and processing data for measuring glycaemia of the individual.

6. The device according to claim 1, wherein the device also comprises a casing, at least partially housing the temperature measuring means, or the means of receiving and processing data for measuring oxygen-pulsed saturation, or the means of receiving and processing data for measuring arterial pressure, or the means of receiving and processing data for measuring electrical heart activity, or the means of receiving and processing data for measuring glycaemia.

7. The device according to claim 3, wherein the device also comprises a casing, at least partially housing the means of receiving and processing data for measuring arterial pressure, the device comprising a motor and/or a pump of an arterial pressure sensor housed at least partially inside the casing.

8. The device according to claim 7, wherein the pump comprises a buffer chamber adapted to limit the noise of the pump when operating.

9. The device according to claim 6, wherein the casing has a length less than or equal to 170 cm or a width less than or equal to 170 cm, for example 120 cm, for example 100 cm, for example 80 cm, or a thickness less than or equal to 170 cm, for example 120 cm, for example 100 cm, for example 80 cm, for example 50 cm, for example 25 cm.

10. The device according to claim 1, wherein the temperature measuring means are for example thermally insulated, preferably by means of a thermally insulating wall, from at least one part of the communication means and/or the processing means.

11. The device according to claim 10, wherein the device also comprises a casing, at least partially housing the temperature measuring means, or the means of receiving and processing data for measuring oxygen-pulsed saturation, or the means of receiving and processing data for measuring arterial pressure, or the means of receiving and processing data for measuring electrical heart activity, or the means of receiving and processing data for measuring glycaemia, wherein the thermally insulating wall extends between a first part and a second part of the casing, the temperature measuring means being arranged in the region of the first part.

12. The device according to claim 1, wherein the device has a mass less than or equal to 5 kg, for example 2 kg, for example 1 kg, for example 500 g.

13. The assembly comprising a device according to claim 1, the assembly also comprising a connector with an electric heart activity sensor, the device being configured such that when the device is physically connected to the electric heart activity sensor via the connector, a battery of the device cannot be charging or the device cannot be supplied by an external power supply.

14. The assembly according to claim 13, the connector comprising:
a first part complementary to a port arranged terminating and/or arranged in the region of an opening of the casing, and
a second blocking part,
such that when the first part is physically connected to said port arranged terminating and/or arranged in the region of the opening of the casing, the second blocking part is arranged so as to prevent any connection to a connection port means having charging means of the battery and/or connection means to the external power supply.
